(19) United States
(12) Patent Application Publication
(10) Pub. No.: US 2002/0058976 A1
(43) Pub. Date: May 16, 2002

(54) Temperature indicating chemotherapy cap

(76) Inventor: Carole Lee, Santa Barbara, CA (US)

Correspondence Address: CHARLES C. LOGAN II, 8282 UNIVERSITY AVENUE, LA MESA, CA, 91941, US

(21) Appl. No.: 09/993,747

(22) Filed: Nov. 14, 2001

Related U.S. Application Data
(60) Provisional application No. 60/249,177, filed on Nov. 15, 2000.

(51) Int. Cl. A61F 7/00

(52) U.S. Cl. 607/110

(57) ABSTRACT

A temperature indicating cap shaped headwear device that is worn on a patient's head while the patient is having an injection-type of chemotherapy treatment. The headwear device has an outer layer of sheet material vertically spaced from an inner layer of sheet material. The outer layer is attached to the inner layer in a quilting pattern that forms individual cells between the outer layer and the inner layer of sheet material. Each of the quilting cells are filled with a temperature indicating material that can be brought to a substantially frozen state causing the material to exhibit a first color. As the material warms to a predetermined higher temperature outside the therapeutic range, the material changes to a second color which provides a visual signal that a new headwear device should be placed on the patients head if further chemotherapy treatment is required.

Applicant hereby claims priority of the provisional patent application serial No. 60/249,177 filed Nov. 15, 2000.

### BACKGROUND OF THE INVENTION

The invention relates to a cap shaped headwear device and more specifically to a temperature indicating chemotherapy cap.

One of the side effects of chemotherapy treatments is that quite often the patients lose most of or all of their hair due to the nature of the strong chemicals used in the treatment for killing cancerous cells. It has been found that by keeping the scalp covered by some type of cold pack during the chemotherapy treatment that loss of hair may be minimized or completely eliminated. The Leong et al U.S. Pat. No. 5,950,234 discloses a cooling pack that is a container having a cooling substance contained within it. The cooling pack is generally constructed such that it can be formed in a bowl to cover the scalp portion of the users head. By covering the scalp with a cooling pack loss of hair is minimized during medical treatments such as chemotherapy. The cooling pack may also be used with headwear such as hats, helmets, and the like. In addition, a headband may have a coolant pack to be worn over the eyebrow area.

Another head cooling device is illustrated in the Tremblay et al U.S. Pat. No. 5,469,579 and its basic purpose is to protect the user from extreme heat conditions that may prompt sunstroke. The Culp U.S. Pat. No. 2,158,571 is directed to an icecap to be worn on the head of a patient during periods of distress incident to common headaches, fevers etc.

The McKay U.S. Pat. No. 5,305,470 illustrates the use of a flexible cold pack that may be inserted into a headband or a wrist band.

It is an object of the invention to provide a novel temperature indicating chemotherapy cap that can be used to minimize hair loss during chemotherapy treatment.

It is also an object of the invention to provide a novel temperature indicating chemotherapy cap that can be reusable by refreezing the coolant contained therein.

It is another object of the invention to provide a novel temperature indicating chemotherapy cap that can be manufactured as a disposable unit, which is used only once.

It is an additional object of the invention to provide a novel temperature indicating chemotherapy cap that is economical to manufacture and market.

It is a further object of the invention to provide a novel temperature indicating chemotherapy cap that has structure to prevent the coolant therein from accumulating against any one end of the cap.

### SUMMARY OF THE INVENTION

The invention relates to a method and device to prevent hair loss as a side effect of chemotherapy. The device is a temperature indicating chemotherapy cap and it may have other uses that may involve the use of other medications or treatments.

The invention is useful when cooling therapy is required within a specific temperature range. The chemotherapy cap is made of a transparent or translucent pre-stitched or pre-formed plastic like material which retains its shape while remaining flexible. The cap is formed in sizes to cover the heads of men, women and children. It is designed to fit from the forehead in the front to the neckline in the back of the patient's head and on the sides of the head to just below the ear. The cap covers all the areas of the head where retention of hair is desired.

The chemotherapy cap is preferably quilted or multi-chambered with each chamber or cell measuring up to 2 inches on each side. The chambers or cells inside the cap are filled with a coolant solution or formula that provides a source of cold temperature that radiates outward and downward toward the scalp. The purpose of the quilting or chambering is to keep the cooling gel solution evenly spread across the cap and patient's head during therapy treatment. The chemotherapy cap is therapeutic when the cooling gel solution is frozen and remains within the temperature range of 28° F. to 36° F. for up to 2 hours. When frozen, the cap is approximately 1 to 1½ inches thick. The cap retains its form so that it covers the patients hair and head in the desired areas.

The cooling gel solution inside the chemotherapy cap is a transparent or translucent formula that radiates its low temperature in a uniform manner throughout the cap. The initial freezing of the gel solution may be done within the cooling cap itself (as with the disposable model) by activating an internal catalyst. Alternatively the gel solution may be frozen by more routine means such as inside a commercial or home freezer. The cooling solution is specifically formulated to stay within the specific and effective temperature range (for example 28° to 36° F.), and for a specific period of time (for example up to two hours) and yet be only 1½ inches thick when in its cold or frozen state. The cooling solution, when frozen, will not hold a temperature of less than 20° F. for safety reasons.

The coolant material is based on liquid crystal technology. One of the most unique properties of this item is the use of liquid crystal technology to indicate the temperature of the cooling gel solution. The cooling gel solution contains liquid-crystal dye, ink and/or liquid crystal additives which react with the temperature of the solution and light-up, glow, or change color when the solution is within the prescribed therapeutic temperature range of from 28 to 36° F. At temperatures above or below this range, the liquid crystals do not react or remain in or return to their original state. Therefore they do not light-up. When the liquid crystal additives react, light-up (glow) or change color the entire cooling gel solution glows or changes color softly and indicates that the solution is within the acceptable temperature range and therefore the product is ready for use. If additional therapy time is required, the cap/solution will indicate the need for a fresh cap through its liquid crystal display. This technology acts as a quality control mechanism by signaling its precise temperature at a glance.

The cap covering the cooling solution may also contain a liquid crystal patch or label at several locations, which will glow or change color when the solution is within the effective temperature range. The patch or label may also be used in place of a liquid crystal containing solution, or both may be present on each device. The cap itself can be manufactured with or impregnated with liquid crystals as a part of the plasticized materials from which the cap is made. With this approach, the entire plasticized cap will glow or change color when the cooling solution that is inserted into it is within the effective temperature range. The liquid crystal properties are in the cap itself, rather than the cooling solution within the cap. These types are normally reusable and require a freezer for initial freezing. An alternative or higher cost model chemotherapy cap could have electronic temperature probes extending into the cooling solution. These probes could be connected to an external monitor which reports the temperature of a cooling solution at different points within the cooling solution bag. The probes can also be attached to external devices or annunciators, which are attached to the cap itself. These devices would include temperature gauges, light emitting diodes, lighting, signal transmitters, etc. These devices would indicate in may different ways whether the coolant solution was within the acceptable temperature range or outside of the range. The temperature cooling solution could also be controlled actively by using the electronic probes and accompanying tubes or shunts which cold or heat is added to the original solution, either chemically or otherwise, to stabilize or change the temperature of the solution within the cap, possibly by computer controls or remote controls.

When using the chemotherapy cap it is important that each patient's hair is cut to a length no longer than 8 to 12 inches long on average (medium length). If the patients hair is excessively thick, hair cut to a shorter length would be recommended. To be effective, the coolant cap must be placed snugly over the head of the patient and must cover all hair that is to be retained. The cap must be placed on the patient's head, when at its effective temperature range, no less than 15 minutes prior to the start of the first infusion of chemotherapy session and must remain in place during its effective temperature range throughout all infusions and until no less than 15 minutes after the final infusion of chemotherapy for the day or session. If a replacement cap is required during therapy, it must be replaced and be at its effective temperature range within 5 minutes of the removal of the existing cap.

It is not necessary to wear the chemotherapy cap other than during periods of chemotherapy is being infused. It is recommended that supervision be available to inspect the cap during infusion to assure that the cap remains within the effective temperature range, as indicated by the liquid crystal properties used in the cap or by any other signaling properties used.

### DESCRIPTION OF THE DRAWINGS

FIG. 1 is a front perspective view illustrating the novel temperature indicating chemotherapy cap;

FIG. 2 is a schematic front perspective view of the novel temperature indicating chemotherapy cap showing portions broken away;

FIG. 3 is a schematic left side elevation view of the novel temperature indicating chemotherapy cap;

FIG. 4 is a schematic front elevation view of the novel temperature indicating chemotherapy cap;

FIG. 5 is a schematic front to rear vertical cross section of the novel temperature indicating chemotherapy cap; and

FIG. 6 is a partial vertical cross section showing the structure of the individual cells of the novel temperature indicating chemotherapy cap.

### DESCRIPTION OF THE PREFERRED EMBODIMENT

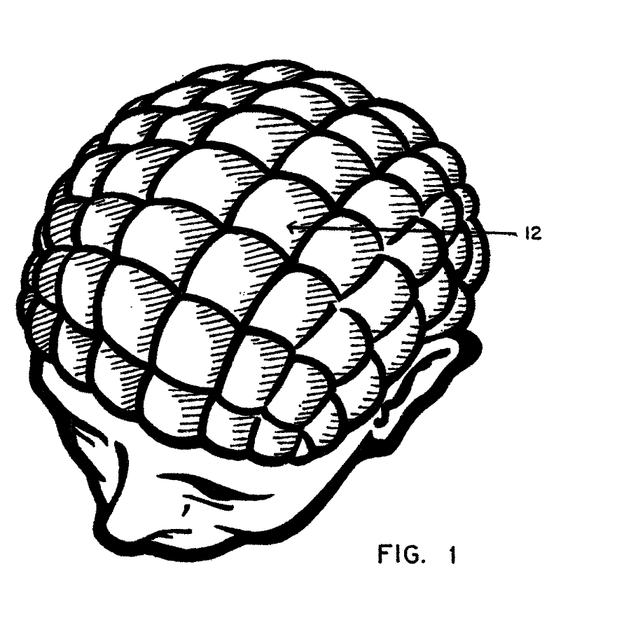

The novel temperature indicating chemotherapy cap will now be described by referring to FIGS. 1-6 of the drawings. The chemotherapy cap is generally designated numeral 10. FIG. 1 is a visual version of what chemotherapy cap 10 might look like. The quilted cells 12 have a distinctive appearance.

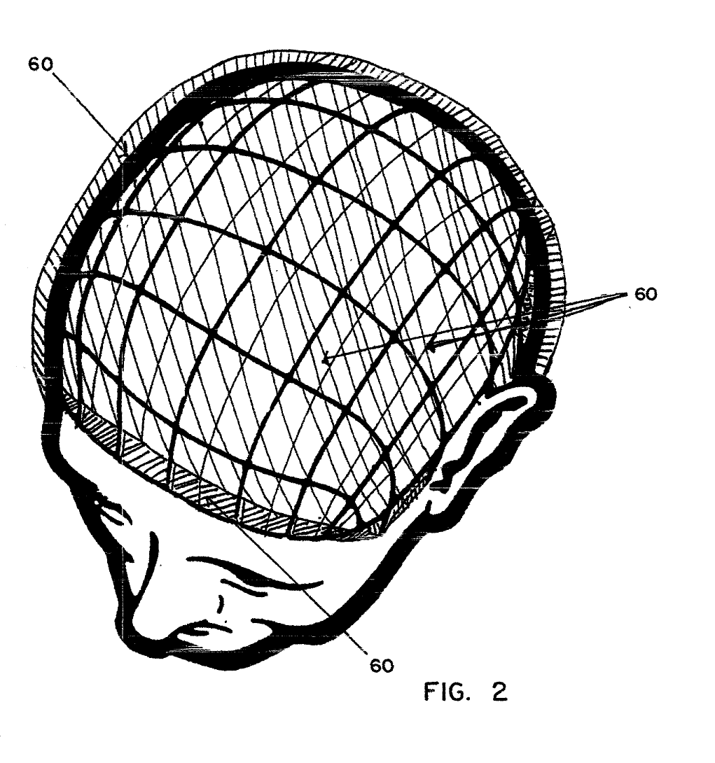

FIGS. 2-6 illustrate a version of what chemotherapy cap 10 looks like from its front end, its rear end, its left side, its right side and what its inner and outer contours would appear like through a longitudinally taken vertical cross section. FIG. 5 is a schematic view showing that chemotherapy cap 10 has a substantially convex outer surface 14, a substantially concave inner surface 16, a front edge 18 and a rear edge 20.

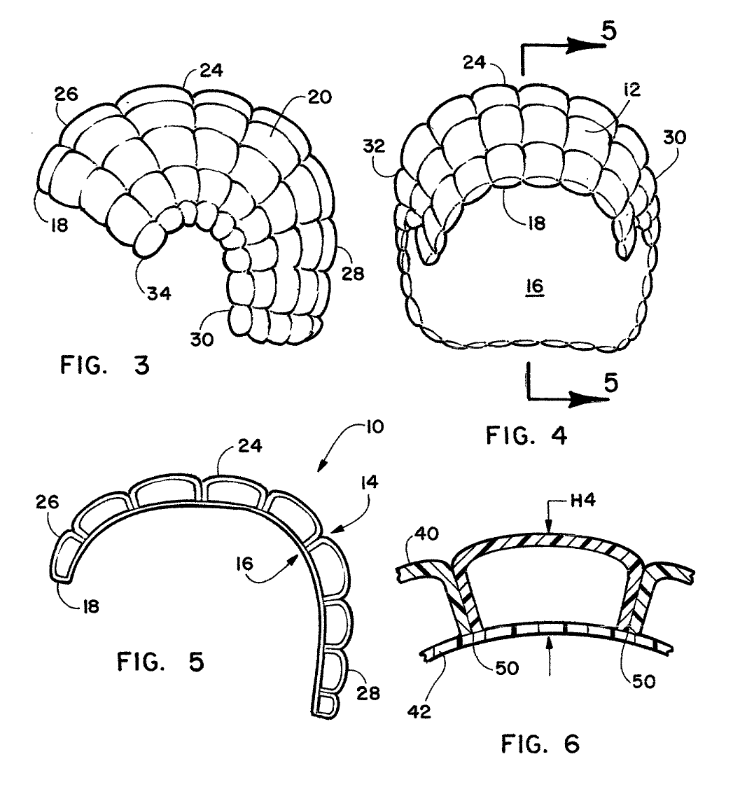

FIGS. 3 and 4 are schematic views showing chemotherapy cap 10 also having a top wall surface 24, a front wall surface 26, a rear wall surface 28, a left side wall surface 30 and a right side wall surface 32. Left side wall surface 30 has a cutout-ear portion 34 and right side wall surface 32 would have a similar cutout-ear portion (not shown).

FIG. 6 is a vertical cross sectional view showing a couple of the individual cells 12. Chemotherapy cap 10 is seen to have a top layer of sheet material 40 that may be plastic, paper or any other suitable material. Bottom layer of sheet material 42 could also be formed of any of the aforementioned materials. The cells are formed by stitches or heat welded seams 50. The cells 12 have a height H1 that is in the range of ½-2 inches. Each cell is filled with a coolant material 60 that may be a gel, a powder, or any other type of material that can be frozen and which would have temperature indicating properties. The coolant material would preferably have a predetermined first color in the T1-T2 range wherein T1 is approximately 28° F. and T2 is approximately 36° F. When the coolant material 60 warms up to a temperature greater than T2 it will exhibit a second predetermined color or no color and thus provide a visual signal that a new headwear device should be placed on the patients head if further chemotherapy treatment is to be continued.

In the claims:

1. A temperature indicating chemotherapy cap comprising:
a cap shaped headwear device to be worn on a person's head while having an injection-type of chemotherapy treatment; said headwear device in its unstressed and unstretched substantially frozen state has a convex outer curvature and a concave inner curvature; said headwear device having a top wall surface, a front wall surface, having a transversely extending front edge, a rear wall surface having a transversely extending rear edge, a left side wall having a bottom edge and a right side wall having a bottom edge;
said headwear device having an outer layer of sheet material vertically spaced from an inner layer of sheet material;
means forming individual cells between said inner layer and said outer layer of sheet material; and
each of said cells having a height H1 at their highest point and said cells being filled with a temperature indicating material that can be cooled to a predetermined temperature in the range of T1 to T2; said material having color changing properties to change to a predetermined first color in the T1-T2 range and said material having color changing properties that change to a predetermined second color when said material warms to a temperature higher than T2 which provides a visual signal that a new headwear device should be placed on the patients head if continued chemotherapy treatment is required.

2. A temperature indicating chemotherapy cap as recited in claim 1 wherein said bottom edges of said left side wall and said right side wall have ear cut-out portions.

3. A temperature indicating chemotherapy cap as recited in claim 1 wherein H1 is in the range of ½ inch-2 inches.

4. A temperature indicating chemotherapy cap as recited in claim 1 wherein T1 is substantially equal to 28° F. and T2 is substantially equal to 36° F.

5. A temperature indicating chemotherapy cap as recited in claim 1 wherein said temperature indicating material is a gel.

6. A temperature indicating chemotherapy cap as recited in claim 1 wherein said temperature indicating material is a powder.

7. A temperature indicating chemotherapy cap as recited in claim 1 wherein said sheet material is plastic.

8. A temperature indicating chemotherapy cap as recited in claim 1 wherein said sheet material is made of a paper material thereby making said headwear device a one-time use disposable product.

9. A temperature indicating chemotherapy cap as recited in claim 1 wherein said means for forming individual quilted cells are stitches.

10. A temperature indicating chemotherapy cap as recited in claim 1 wherein said means for forming individual quilted cells are heat welded seams.

11. A temperature indicating chemotherapy cap comprising:
a cap shaped headwear device to be worn on a person's head while having an injection-type of chemotherapy treatment; said headwear device in its unstressed and unstretched substantially frozen state has a convex outer curvature and a concave inner curvature; said headwear device having a top wall surface, a front wall surface, having a transversely extending front edge, a rear wall surface having a transversely extending rear edge, a left side wall having a bottom edge and a right side wall having a bottom edge;
said headwear device having an outer layer of sheet material vertically spaced from an inner layer of sheet material;
means forming individual cells between said inner layer and said outer layer of sheet material; and
each of said cells having a height H1 at their highest point and said cells being filled with a fluid that can be cooled to a predetermined temperature in the range of T1 to T2; a plurality of temperature sensitive patches on a plurality of said cells, said temperature sensitive patches having color changing properties to change to a predetermined first color in the T1-T2 range and said temperature sensitive patches having color changing properties that change to a predetermined second color when said fluid warms to a temperature higher than T2 which provides a visual signal that a new headwear device should be placed on the patients head if continued chemotherapy treatment is required.

12. A method of using said temperature indicating chemotherapy cap as recited in claim 1 comprising the following steps:
a) placing said chemotherapy cap on a patient's head for at least 15 minutes;
b) giving the patient an injection chemotherapy treatment for the amount of time required by the patient's doctor;
c) keep said chemotherapy hat on the patient's head at least 15 minutes after completion of said chemotherapy treatment.
